(19) United States
(12) Patent Application Publication
(10) Pub. No.: US 2003/0108876 A1
(43) Pub. Date: Jun. 12, 2003

(54) Method for the application of FTMS to drug testing

(76) Inventor: Johnny Paul Speir, Merrimack, NH (US)

Correspondence Address: Ward & Olivo, 708 Third Ave, New York, NY, 10017, US

(21) Appl. No.: 10/014,279

(22) Filed: Dec. 11, 2001

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

The present invention is a method for analyzing a biological sample using a Fourier Transform Mass Spectrometer comprising the steps of ionizing a sample to produce sample ions; introducing said sample ions into an analysis region of said Fourier Transform Mass Spectrometer; analyzing said sample ions to determine the molecular weight and abundance of said sample ions; utilizing said molecular weight to determine the empirical formula of each species of said sample; and identifying each of said species by comparing said empirical formula to a database of formulas for known molecules.

### TECHNICAL FIELD OF THE INVENTION

The present invention relates generally to the detection and discovery of drugs. More specifically, the present invention relates to a Fourier transform mass spectrometry (FTMS) system or similar devices to utilize ultra high resolution and sensitivity to detect drug metabolites in cell extracts. This allows the present invention to detect drug metabolites, in vitro, under high throughput conditions, thereby providing a means for the rapid screening of drug dosed, biological samples. The disclosed method will be particularly useful in high speed testing of experimental pharmaceutical and other chemical compounds.

### BACKGROUND OF THE PRESENT INVENTION

Fourier transform mass spectrometers can be adapted mainly for general organic analysis to identify unknown components. Generally, a transmission unit supplying high-frequency electric field, mounted to the Fourier transform mass spectrometer forms an electric field for ionizing a gaseous sample. This transmission unit sweeps a region of resonant frequency corresponding to a region of mass to be measured at a high speed so as to excite all kinds of ions.

The field of drug discovery and testing is constantly changing due to rapidly evolving technology in the field. Recent advances include the rational and combinatorial design of synthetic molecules as potential bioactive agents (e.g., ligands, agonists, antagonists, and inhibitors) and the identification, and mechanistic and structural characterization of their biological targets (e.g., polypeptides, proteins, and/or nucleic acids). The key to understanding and treating diseases lies in these areas of drug design and structural biology. However, several problems exist, including the difficulty in elucidating the structure of targets, the colossal numbers of compounds that need to be screened, the need to analyze structural similarities and differences between these compounds, correlating structural features to activity and binding affinity, and the fact that small structural changes can lead to large effects on biological activities of compounds. In addition to screening, a further problem resides in the bottleneck created by the even more important testing of compounds of interest. The days of injecting rats and making physical observations over the course of weeks or months is coming to a close.

Traditionally, the process of drug discovery and characterization is slow due to its expensive and lengthy nature. When the process is implemented using natural products, the individual components of the natural extracts must be separated into pure compounds prior to evaluation. This process is slow and expensive due to its extensive nature. Further, all such compounds have to be carefully analyzed and characterized before being screened in-vitro. These in-vitro screens include the evaluation of compounds for their binding affinity to a target, the competition for a ligand binding site, or the efficacy at a target as determined by inhibition, cell proliferation, activation or antagonism end points. All of these phases of drug design and screening slow the process of drug discovery, and therefore several approaches to lessen the time and expense of these processes have been recently implemented.

For example, the time for completion of the drug discovery process is being shortened by the generation of large libraries, of compounds. Through these libraries, the strategy of detection and characterization of compounds has shifted from merely selecting drug leads from detecting compounds that are individually created and tested to the simultaneous screening of large collections of compounds. The collections of compounds may be utilized from natural sources (Stemberg et al., Proc. Natl. Acad. Sci. USA, 1995, 92, 1609-1613) or generated by such scientific methods as combinatorial chemistry (Ecker and Crooke, BioTechnology, 1995, 13, 351-360 and U.S. Pat. No. 5,571,902, incorporated herein by reference). These libraries are collections of individual, well-characterized compounds synthesized, through a number of processes (e.g., high throughput, parallel synthesis, split mix method, or a combination of combinatorial methods).

The screening of such libraries typically involves a binding assay to determine the extent of ligand-receptor interaction (Chu et al., J. Am. Chem. Soc., 1996, 118, 7827-35). The ligand or the target receptor is often immobilized onto a surface (e.g., a polymer bead or plate). Following detection of a binding event, the ligand can be released and identified. However, solid phase screening assays can be rendered difficult by non-specific interactions.

Screening of such libraries can be performed using solid-phase, solution methods or other processes. Regardless of process used, the identification of those components of the library which bind to a target in a rapid and effective manner is difficult. Not coincidentally, such components are of the greatest interest when detecting and characterizing compounds.

Other approaches to assisting the understanding of a structure of biopolymeric and other therapeutic targets have been developed to attempt to hasten the process of drug discovery and characterization. These processes include the sequencing of proteins and nucleic acids (Smith, in Protein Sequencing Protocols, Humana Press, Totowa, N.J., 1997; Findlay and Geisow, in Protein Sequencing: A Practical Approach, IRL Press, Oxford, 1989; Brown, in DNA Sequencing, IRL Oxford University Press, Oxford, 1994; Adams, Fields and Venter, in Automated DNA Sequencing and Analysis, Academic Press, San Diego, 1994); elucidating the secondary and tertiary structures of such biopolymers via NMR (Jefson, Ann. Rep. in Med. Chem., 1988, 23, 275; Erikson and Fesik, Ann. Rep. in Med. Chem., 1992, 27, 271-289), X-ray crystallography (Erikson and Fesik, Ann. Rep. in Med. Chem., 1992, 27, 271-289); and the use of computer algorithms to attempt the prediction of protein folding (Copeland, in Methods of Protein Analysis: A Practical Guide to Laboratory Protocols, Chapman and Hall, New York, 1994; Creighton, in Protein Folding, W. H. Freeman and Co., 1992). Experiments such as ELISA (Kemeny and Challacombe, in ELISA and other Solid Phase Immunoassays: Theoretical and Practical Aspects; Wiley, N.Y. 1988) and radioligand binding assays (Berson and Yalow, Clin. Chim. Acta, 1968,22, 51-60; Chard, in “An Introduction to Radioimmunoassay and Related Techniques,” Elsevier press, Amsterdam/N.Y., 1982), the use of surface-plasmon resonance (Karlsson, Michaelsson and Mattson, J. Immunol. Methods, 1991, 145, 229; Jonsson et al., Biotechniques, 1991, 11, 620), and scintillation proximity assays (Udenfriend, Gerber and Nelson, Anal. Biochem., 1987, 161, 494-500) are currently being utilized to understand the nature of the receptor-ligand interaction.

Moreover, several tools and techniques exist for the structural elucidation of biologically interesting targets, for the determination of the strength and stoichiometry of target-ligand interactions, and for the determination of active components of combinatorial mixtures. For example, for the sequencing of biological targets such as proteins and nucleic acids (e.g. Smith, in Protein Sequencing Protocols, 1997 and Findlay and Geisow, in Protein Sequencing: A Practical Approach, 1989) cited previously.

X-ray crystallography is also a very powerful technique that allows for the determination of some secondary and tertiary structures of biopolymeric targets (Erikson and Fesik, Ann. Rep. in Med. Chem., 1992, 27, 271-289). However, X-ray crystallography is an expensive and very difficult process. Crystallization of biopolymers is especially difficult to perform at an adequate resolution. Further confusing the utility of X-ray crystal structures in the drug discovery process is the inability of such a process to reveal any insights into the solution-phase. This excludes many biologically relevant structures of the targets of interest.

The field of chemical synthesis of compounds for high-throughput biological screening has also seen numerous technological advances. Combinatorial chemistry, computational chemistry, and the synthesis of large collections of mixtures of compounds or of individual compounds have all made the rapid synthesis of large numbers of compounds for in vitro screening easier. One of the steps in the identification of bioactive compounds requires the determination of binding affinity of test compounds for a desired biopolymeric or other receptor (i.e., a specific protein, nucleic acid or combination thereof). Because using combinatorial chemistry gives one the ability to synthesize large numbers of compounds for in vitro biological screening, the determination of binding affinity of those test compounds is more difficult. Also, since combinatorial chemistry generates large numbers of compounds or natural products, the need for methods which allow rapid determination of those test compounds which are most active or which bind with the highest affinity to a receptor target exists.

When screening combinatorial mixtures of compounds, an active pool is identified, deconvoluted into individual members using a resynthesis technique, and then the active members are identified through analysis of the discrete compounds. Current techniques and methods for the study of combinatorial libraries against a variety of biologically relevant targets are tedious and expensive. The multi-step character, and low sensitivity of the above technologies are also existing problems when utilizing currently available methods. Another drawback to present solutions includes the inability to provide the most relevant structural information, such as the structure of a target in solution. Instead the method could provide insights into target structures that may only exist in a solid phase. Typically, current methods cannot provide a convenient way to the deconvolute and identify active members of libraries without having to perform tedious re-syntheses and re-analyses of discrete members of pools or mixtures.

However, new methods for screening and identifying complex chemical libraries (especially combinatorial libraries) are being developed. For example, Crooke et al. U.S. Pat. No. 6,329,146 discloses methods for the determination of the structure of biomolecular targets, as well as the site and nature of the interaction between ligands and biomolecular targets. Crooke also provides methods for screening ligand or combinatorial libraries of compounds against one or more than one biological target molecules.

While advances are clearly being made in the detection, screening and identification of bioactive compounds and new drug candidates, one area has apparently been ignored. Specifically, the technological improvements in these areas of drug development have not been applied to drug testing. The present invention seeks to remedy this situation.

### SUMMARY OF THE INVENTION

The present invention is a method for analyzing a biological sample using a Fourier Transform Mass Spectrometer comprising the steps of ionizing a sample to produce sample ions; introducing said sample ions into an analysis region of said Fourier Transform Mass Spectrometer; analyzing said sample ions to determine the molecular weight and abundance of said sample ions; utilizing said molecular weight to determine the empirical formula of each species of said sample; and identifying each of said species by comparing said empirical formula to a database of formulas for known molecules.

It is an object of the present invention to utilize the high mass accuracy of the FTMS system or similar devices to determine the elemental formulae of a test compound. Searching elemental formulae, with constraints, can help correctly identify compounds detected in test mixtures.

It is another object of the present invention to utilize multiple stages of mass spectrometry for the structure elucidation of compounds present in the biological matrix. It is often insufficient to rely solely on molecular formulae searches for structure determinations. Therefore it may be necessary to perform multiple stages of mass spectrometry to dissociate a detected component into smaller fragments.

It is yet another object of the present invention to perform multiple stages of mass spectrometry rapidly (in keeping with high throughput requirements) using photodissociation.

It is yet another object of the present invention to utilize atmospheric pressure ionization (API) modes of operation to maximize the detection coverage of cellular contents.

It is yet another object of the present invention to utilize FTMS technology to test drugs to continuously analyze drug interactions at high throughput.

Other objects, features, and characteristics of the present invention, as well as the methods of operation and functions of the related elements of the structure, and the combination of parts and economies of manufacture, will become more apparent upon consideration of the following detailed description with reference to the accompanying drawings, all of which form a part of this specification.

### BRIEF DESCRIPTION OF THE DRAWINGS

A further understanding of the present invention can be obtained by reference to a preferred embodiment set forth in the illustrations of the accompanying drawings. Although the illustrated embodiment is merely exemplary of systems for carrying out the present invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the present invention, reference is now made to the following drawings in which:

FIG. 1 depicts an FTMS spectrum of a test sample reacting to a drug showing detected metabolic products.

FIG. 2 depicts elemental formulae searches for detected metabolic products observed in the spectrum of FIG. 1.

FIG. 3 depicts expanded regions of the spectrum shown in FIG. 1.

FIG. 4 depicts mass spectra of treated serum samples illustrating the ability of the present invention to identify cellular changes.

### DETAILED DESCRIPTION OF THE PREFERRED EMBODIMENTS

As required, a detailed illustrative embodiment of the present invention is disclosed herein. However, techniques, systems and operating structures in accordance with the present invention may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein which define the scope of the present invention. The following presents a detailed description of a preferred embodiment (as well as some alternative embodiments) of the present invention.

The present invention relates generally to the use of mass spectrometry in the field of biological detection and characterization. More specifically, while the monitoring of predicted drug metabolites in biological fluids is practiced throughout the industry, the monitoring and characterization of all detectable chemical changes in a cell following drug dosage has never before been accomplished. The method of the present invention includes analyzing a biological sample using a Fourier Transform Mass Spectrometer comprising the steps of ionizing a sample to produce sample ions; introducing said sample ions into an analysis region of said Fourier Transform Mass Spectrometer; analyzing said sample ions to determine the molecular weight and abundance of said sample ions; utilizing said molecular weight to determine the empirical formula of each species of said sample; and identifying each of said species by comparing said empirical formula to a database of formulas for known molecules.

The method of the present invention allows a test sample that is affected by several dosages of drugs in a high throughput to be analyzed continuously to determine the effects of the drug dosages.

Mass spectrometry is a powerful analytical tool for the study of molecular structure and interaction between small and large molecules. An accurate measurement of a sample's molecular weight may be obtained quickly, whether the sample's molecular weight is several hundred, or in excess of a hundred thousand Daltons (Da). Mass spectrometry can elucidate significant aspects of important biological molecules. One reason for the utility of mass spectrometers as analytical tools is the availability of a variety of different methods, instruments and techniques which can provide different pieces of information about the samples.

As discussed above, Fourier transform mass spectrometry (FTMS) is an especially useful technique because of its high accuracy and resolution. Significantly, other similar devices yielding high accuracy and resolution can be utlized eith the present invention as well. FTMS can measure the mass of compounds with such accuracy and resolution superior to other mass spectrometers. Further, it may be used to obtain high resolution mass spectra of ions generated by any other ionization technique. The basis for FTMS is ion cyclotron motion, which is the result of the interaction of an ion with a unidirectional magnetic field. The mass-to-charge ratio of an ion (m/q or m/z) is determined by a FTMS instrument by measuring the cyclotron frequency of the ion. The insensitivity of the cyclotron frequency to the kinetic energy of an ion is one of the fundamental reasons for the very high resolution achievable with FTMS.

Tandem mass spectrometry has been found to be a useful tool for determining the structure of biomolecules. It is known in the art that both small and large (>3000 kbase) RNA and DNA may be transferred from solution into the gas phase as intact ions using electrospray techniques. Further it is known, to those skilled in the art that these ions retain some degree of their solution structures as ions in the gas phase; this is especially useful when studying noncovalent complexes of nucleic acids and proteins, and nucleic acids and small molecules by mass spectrometric techniques. See U.S. Pat. No. 6,329,146 to Crooke, at al.

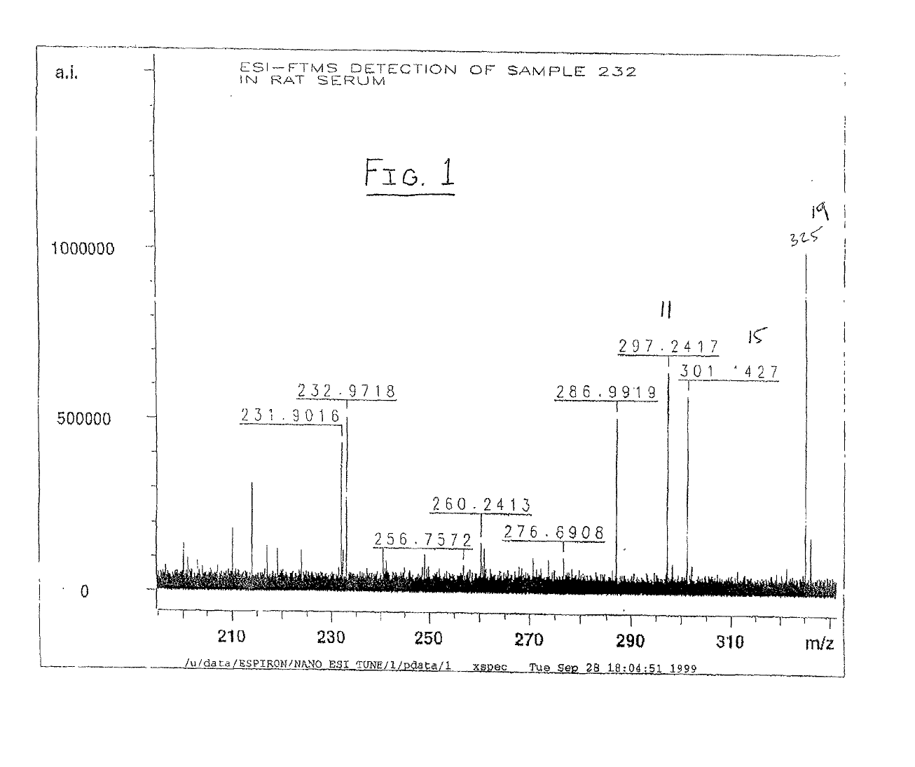

Referring first to FIG. 1, shown is an FTMS spectrum of a test sample reacting to a drug (Drug Sample 232) showing detected metabolic products. A rat serum sample was affected by Drug Sample 232 to produce unpredicted events. Shown are unidentified metabolic products (11, 15 and 19) of the dosed drug. By utilizing the FTMS system, or any other similar device, the present invention uses a high mass accuracy to determine the molecular weight of the metabolic compounds. To this end, the compound can be accurately determined. For example, the compound C₁₃H₂₀N₂O₃ has a molecular weight of 252.1468 Da, while the compound C₁₄H₂₄N₂O₂ has a molecular weight of 252.1832 Da, a difference of just 0.0364 Da. FTMS is capable of routinely providing such mass measurements with an error less than 3 ppm. Therefore selectivity can be achieved for ions which are separated by extremely small mass differences. In an alternative embodiment, an intelligent database can be created which “learns” as new cellular changes are detected.

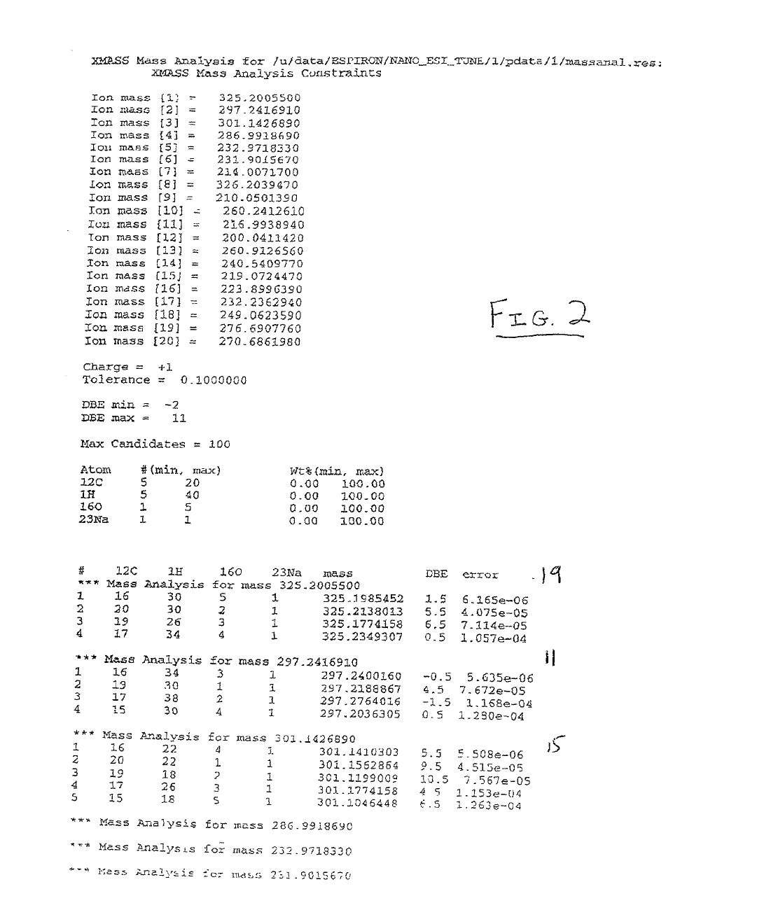

Referring now to FIG. 2, shown are the elemental formulae searches for the detected metabolic products observed in the spectrum of FIG. 1. Shown is unidentified element 11 with molecular weight 297.2416910. The elemental formulae search shows that unidentified element 11 has been calculated to be C₁₆H₃₄O₃+Na. The error associated with the calculation is shown as 5.653×10⁻⁶. Similarly, the elemental formulae search shows that unidentified element 15 (molecular weight 301.1426890) has been calculated to be C₁₆H₂₂O₄+Na. The error associated with this calculation is shown as 5.508×10⁻⁶. Finally, the elemental formulae search shows that unidentified element 19 (molecular weight 325.2005500) has been calculated to be C₁₆H₃₀O₅+Na. The error associated with that calculation is shown as 6.156×10⁻⁶.Therefore, by utilizing the FTMS system's high mass accuracy, the present invention can immediately target and characterize the changes to a sample serum caused by a drug.

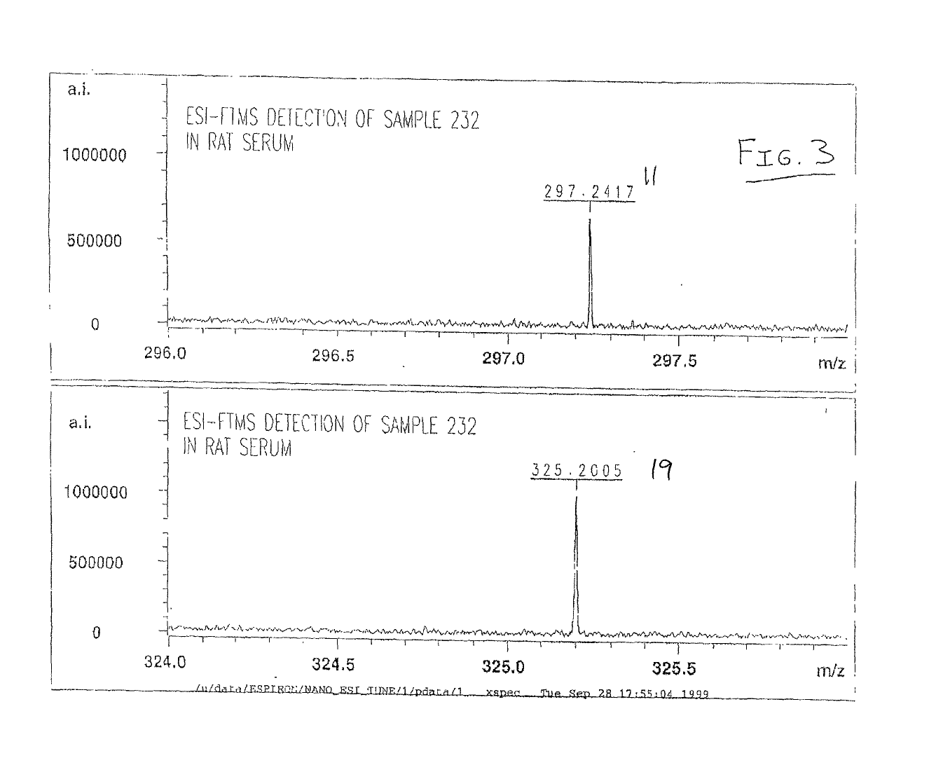

Referring now to FIG. 3, shown are expanded regions of the spectrum shown in FIG. 1, showing unidentified compounds 11 and 19.

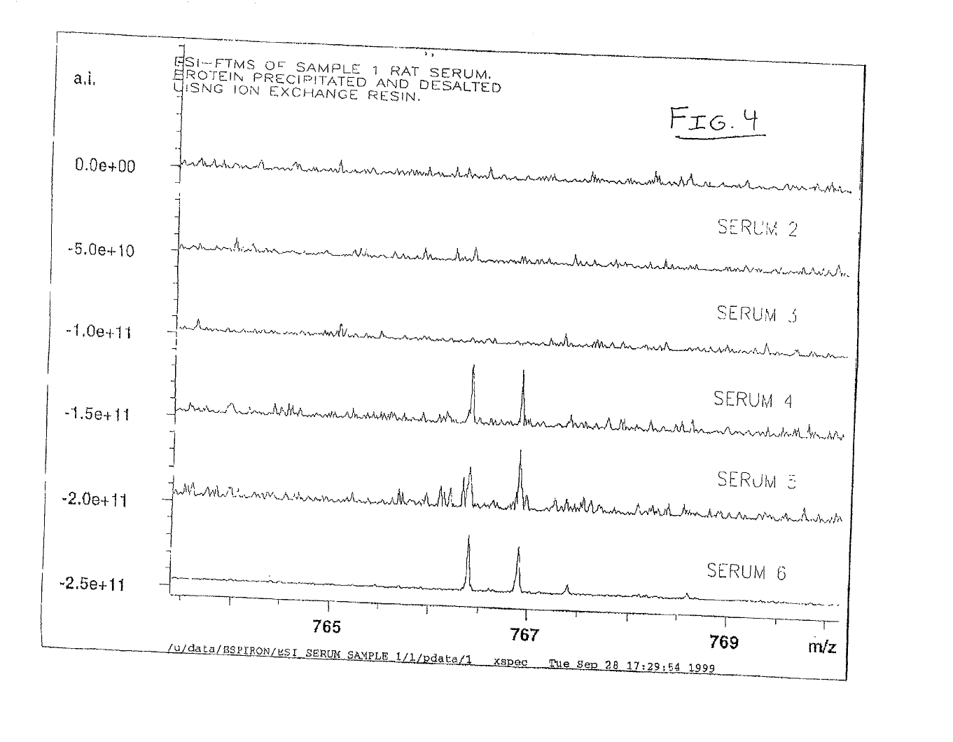

Referring finally to FIG. 4, shown are mass spectra of treated serum samples illustrating the ability of the present invention to identify cellular changes.

The present invention as disclosed represents a dramatic advance in the field of drug testing. This advance is represented by several factors including sensitivity, time and expense. Presently, drug testing often involves the injection of a drug into a test species and observing the effects as they appear over a period of days, weeks or months. The present invention allows drug testing thorough injection of a drug into test cells, and immediately monitoring the effects.

The present invention allows for the immediate observation of test cells because of the increased sensitivity of the FTMS apparatus. FIGS. 1 and 2 show that once test cells have been subjected to a drug, the resulting compounds can immediately be identified using FTMS precision to within 3 ppm. The FTMS apparatus weighs the resulting compounds as depicted in FIG. 1 and then accurately identifies the compound as shown in FIG. 2. This allows the constant and immediate monitoring of the effects of a drug on the test cells.

Inherent in the precise analysis which the present invention makes possible is the ability to conduct drug testing in a matter of minutes, instead of weeks or months. Instead of injecting a test species and observing any evident effects on the species over a long period of time, researchers can inject test cells with a drug and observe the interactions and effects immediately. This allows researchers to test and identify all changes in cellular composition, instead of merely locating and verifying the anticipated changes in an injected species, as was done previously for all drug testing. The present invention also allows the ability to observe all effects of a drug on test cells, as opposed to merely the evident effects one could observe from a test species over time. Additionally, the ease and speed with which one can make observations using the method of the present invention allows a researcher to maintain throughput. Rather than waiting for weeks for observations, a researcher can observe cellular reactions, adjust parameters, view results and continue experimentation in a matter of minutes.

The ease and immediate effectiveness of the present invention reduces the cost of the overall drug testing. There is no longer a need to house and care for several series of test species as well as corresponding control species in order to determine the differences between species injected with the drug and the control group. Diminished space requirements, ease of testing procedure, and reduction of total test monitoring time all contribute to a decreased cost of testing.

In alternative embodiments of the present invention, multiple stages of mass spectrometry (MSⁿ) may be utilized for the structure elucidation of compounds. MSⁿ may be used in combination with elemental formulae searches of fragmented ions to determine the structure of interesting, unknown metabolites. It is often insufficient to rely solely on molecular formulae searches for structure determinations (i.e., geometric isomers, complete unknowns, etc.). Therefore it may be necessary to perform multiple stages of mass spectrometry to dissociate a detected component into smaller fragments, with each fragment being unique to a particular moiety of the molecule. Msⁿ can be performed rapidly (thereby keeping with high throughput requirements) using photodissociation.

In yet another alternative embodiment of the present invention, the use of atmospheric pressure ionization (API) modes of operation can help maximize the detection coverage of cellular contents. Specifically, because cells contain both polar and non-polar compounds, as well as acidic and basic compounds, different API modes would be necessary. For example, positive/negative electrospray would maximize the detection coverage of polar compounds. Similarly, positive/negative atmospheric pressure chemical ionization would maximize the detection coverage of non-polar species. Therefore, in alternative embodiments of the present invention, operation of all four API modes can maximize the detection coverage of metabolic changes.

While the present invention has been described with reference to one or more preferred embodiments, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

What is claimed is:

1. A method for analyzing a complex biological sample using a Fourier Transform Mass Spectrometer (FTMS), said method comprising the steps of:
a. ionizing a sample to produce sample (molecular) ions;
b. introducing said ions into an analysis region of said FTMS;
c. analyzing said ions to determine the molecular weight and abundance of said ions;
d. utilizing said molecular weight to determine the empirical formula of each species of said sample; and
e. identifying each said species by comparing said empirical formula to a database of formulas for known molecules.

2. A method according to claim 1, wherein said determining of the molecular weight is performed with an accuracy sufficient to identify empirical formula of said ions.

3. A method according to claim 1, wherein said database of known molecules is updated with said determined molecular structures.

4. A method for analyzing a complex biological sample using a Fourier Transform Mass Spectrometer (FTMS), said method comprising the steps of:
a. ionizing a sample to produce sample (molecular) ions;
b. introducing said ions into an analysis region of said FTMS;
c. analyzing said ions to determine the molecular weight and abundance of said ions;
d. determining the molecular structure of each species by multiple stages of mass spectrometry; and
e. producing a profile of the sample showing structure and concentration data for each species.

5. A method according to claim 4, wherein said determining of the molecular weight is performed with an accuracy sufficient to identify empirical formula of said ions.

6. A method for analyzing a complex biological sample using a Fourier Transform Mass Spectrometer (FTMS), said method comprising the steps of:
a. ionizing the sample to produce sample precursor ions;
b. introducing said ions into the analysis region of said FTMS;
c. analyzing said ions to determine the molecular weight, the abundance and the empirical formula of said ions;
d. fragmenting said sample precursor ions to produce fragment ions;
e. determining the molecular weight, the abundance and empirical formula of said fragment ions;
f. determining the structure of said fragment ions by comparing said empirical formulas of said fragment ions to a database of fragments with known structure;
g. combining said structures of said fragment ions to determine the precursor ion structure for each species in said sample; and
h. producing a profile of said sample showing structure and concentration data for selected species of said sample.

7. A method according to claim 6, wherein said determining of the molecular weight is performed with an accuracy sufficient to identify empirical formula of said ions.

8. A method according to claim 6, wherein said fragmenting is performed using photodissociation.

9. A method for analyzing a complex biological sample utilizing Fourier Transform Mass Spectrometry (FTMS), said method comprising the steps of:
a. ionizing polar molecules using positive and negative electrospray to produce sample (molecular) ions;
b. introducing said ions into an analysis region of said FTMS;
c. analyzing said ions to determine the molecular weight and abundance of said ions;
d. utilizing said molecular weight to determine the empirical formula of each species of said sample; and
e. identifying each said species by comparing said empirical formula to a database of formulas for known molecules.

10. A method according to claim 9, wherein said determining of the molecular weight is performed with an accuracy sufficient to identify empirical formula of said ions.

11. A method according to claim 9, wherein said database of known molecules is updated with said determined molecular structures.

12. A method for analyzing a complex biological sample utilizing Fourier Transform Mass Spectrometry (FTMS), said method comprising the steps of:
a. ionizing non-polar molecules using positive and negative ion atmospheric pressure chemical ionization to produce sample (molecular) ions.
b. introducing said ions into an analysis region of said FTMS;
c. analyzing said ions to determine the molecular weight and abundance of said ions;
d. utilizing said molecular weight to determine the empirical formula of each species of said sample; and
e. identifying each said species by comparing said empirical formula to a database of formulas for known molecules.

13. A method according to claim 12, wherein said determining of the molecular weight is performed with an accuracy sufficient to identify empirical formula of said ions.

14. A method according to claim 12, wherein said database of known molecules is updated with said determined molecular structures.
